(19) United States
(12) Patent Application Publication
(10) Pub. No.: US 2012/0208202 A1
(43) Pub. Date: Aug. 16, 2012

(54) Method for the Reduction of Biological Sampling Errors by Means of Image Processing

(75) Inventors: Andrew Wheeler, Saco, ME (US); Roger N. Piasio, Cumberland Foreside, ME (US); Christopher Turmel, Portland, ME (US)

(73) Assignee: Alere Scarborough, Inc., Scarborough, ME (US)

(21) Appl. No.: 13/236,038

(22) Filed: Sep. 19, 2011

Related U.S. Application Data
(60) Provisional application No. 61/383,918, filed on Sep. 17, 2010.

(51) Int. Cl. G01N 33/53

(52) U.S. Cl. 435/7.1

(57) ABSTRACT

The present invention relates to methods and devices for reducing biological sampling errors by means of image processing. Image processing techniques are used to determine the volume of sample added to a device, such as a diagnostic test, and to correct for user error in sampling techniques.

### BACKGROUND

Biological sampling error is a significant problem in point of care diagnostics. A user must be able to ensure that the proper amount of a sample, for example a blood sample, is collected and applied to a diagnostic test in order for the test to be performed correctly and accurately. Complicated techniques and equipment are not available for users to determine if the proper amount of sample has been collected and applied to the test, so the user is forced to use his or her judgment on whether or not the correct amount of sample has been collected. Too little or too much sample can have profound effects on assays where the amount of sample has a direct impact on the signal that is generated.

### SUMMARY

The purpose of the invention is to account for errors in sampling, and to correlate a generated signal response accordingly. Using image processing to determine the volume of sample added to a diagnostic test device allows for user error (which is inevitable), but corrects for user error, so that the precision of the assay is not affected.

In one embodiment, sample is applied to a sample application zone of a diagnostic test device. An imaging device, for example a camera, captures an image of a sample application zone. The image that results is then processed to determine intensity of color (for colored samples such as blood or urine) or gray scale differences between a wet and dry surface. The image is also processed to determine the diameter and area of a spot that is generated by adding the sample to the device. When more sample is added, the resulting spot is larger.

The resulting information captured in processing the image can be used during the manufacturing of the device lot and entered as parameters in a table. For example, if an unknown volume is imaged and processed and the spot area is determined to be 350 units, software may be used to look up the linear curve of the diameter area and determine that this correlates to a volume of 14.2 μL. This data is then used in another table that correlates volume of sample to a dose response curve for assay analyte signal, and outputs the correct offset to account for the sample difference.

### BRIEF DESCRIPTION OF THE DRAWINGS

FIG. 1 is an image of a blood spot from 50 μL of blood.

FIG. 2 is an image of a blood spot from 20 μL of blood.

FIG. 3 is an image of a blood spot from 40 μL of blood.

FIG. 4 is a chart of spot area vs. μL of blood placed onto a membrane

FIG. 5 is a drawing of a 5 μL spot with electrode placement.

FIG. 6 is a drawing of a 20 μL spot with electrode placement.

FIG. 7 is an image of blood spots on lateral flow test strips ranging from 15 μL to 40 μL.

FIG. 8 is a chart showing area versus amount of blood dispensed.

FIG. 9 is a chart showing perimeter versus amount of blood dispensed.

FIG. 10 is a chart showing IOD versus amount of blood dispensed.

FIG. 11 is a chart showing area (polygon) versus amount of blood dispensed.

FIG. 12 is an image of a test strip.

FIG. 13 is an image of a capillary tube.

FIG. 14 is a computer screen shot of the analysis of the capillary tube of FIG. 13.

### DETAILED DESCRIPTION

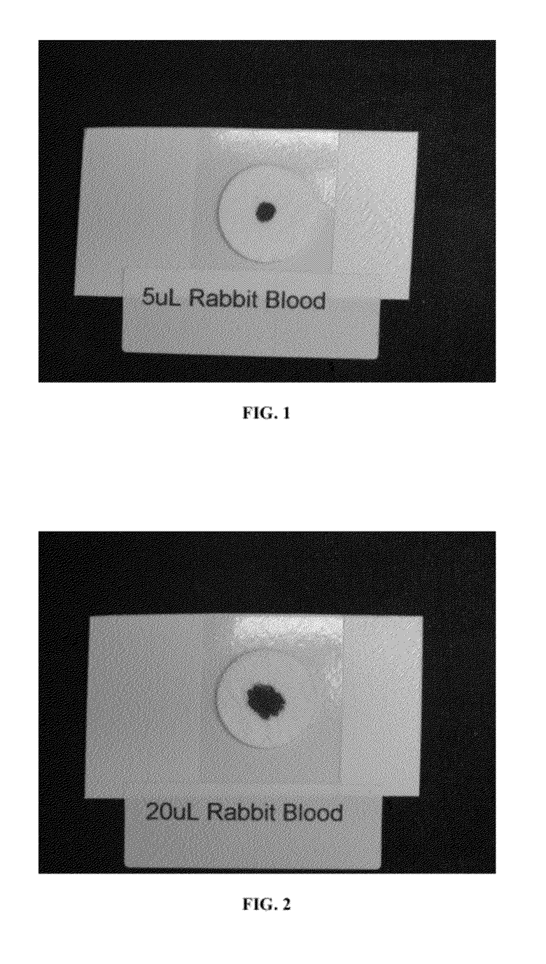

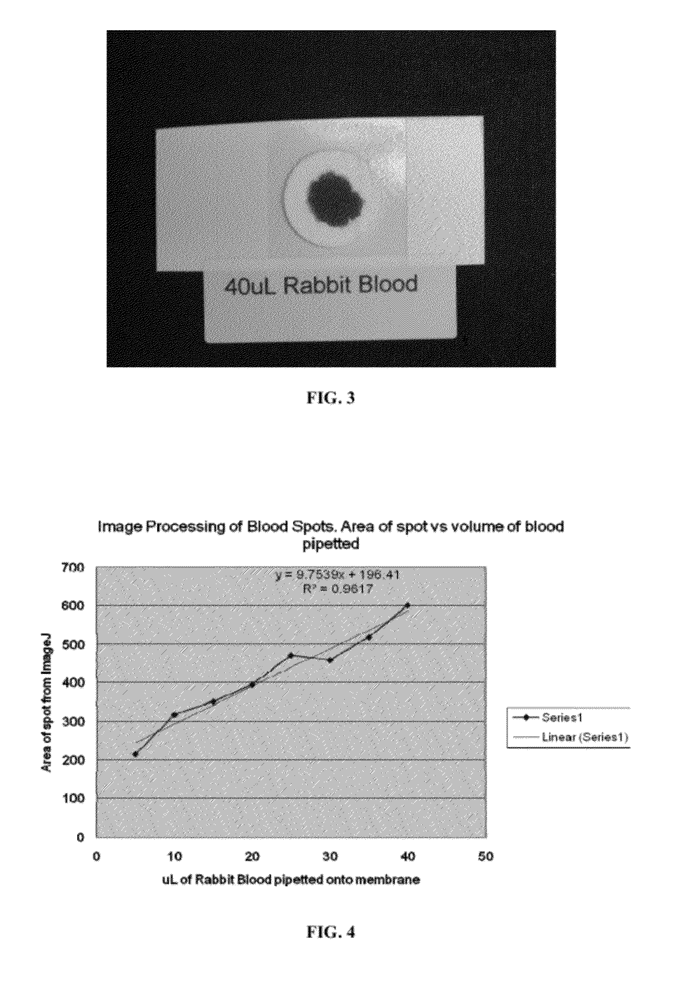

In the examples described below, Whatman VF2 blood separation membranes were used. The material was backed onto a G&L lateral flow polyester backing material so that the flow of liquid into the membrane would be contained to the surface level, with no wicking onto the back side. Various amounts of rabbit whole blood was added to the blood separation pad using a pipette. A digital camera was then used to take images of the resulting blood spots. FIGS. 1-3 show the blood spots produced by 5 μL of blood, 20 μL of blood, and 40 μL of blood, respectively.

The images are processed to determine the area of the spot. In the examples shown in FIGS. 1-4, an image processing program called imageJ was used to determine the area of the spot. The resulting area was plotted against pipette volume to determine the correlation. FIG. 4 shows a chart of spot area vs. μL of blood placed onto a membrane using a pipette. This chart shows that as the the area of the spot (measured by diameter of the colored spot) increases linearly with the amount of liquid that is pipetted onto the membrane, more advanced software and algorithms will only make the measurement more precise. An equation for finding an unknown volume could be used if the area of the colored spot is known. In the examples shown in FIGS. 1-4, reflective light was used, with light shining on the blood separation pad and reflecting off of the pad and into the camera.

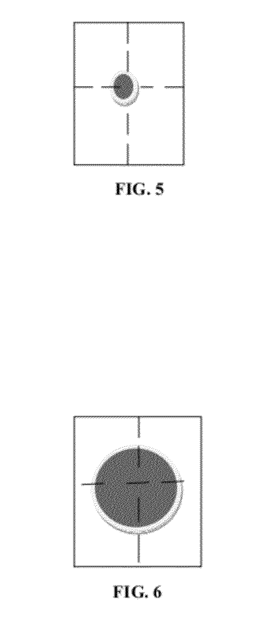

FIGS. 5 and 6 show an alternate method of analysis which is based on a digital test device. One example of a digital device is the Clearblue Easy digital pregnancy test. This test employs electrodes that detect moisture in order to notify the electronics that the assay has begun. One could employ a grid of electrodes across the width and height of the sample application zone. The dotted lines in FIGS. 5 and 6 represent electrodes. A measurement of the electrodes that have detected moisture can reveal the diameter of the spot. The number of electrodes that are turned on (i.e. detect moisture) indicates the size and shape of the spot. This information can then be fed back into the system as described above.

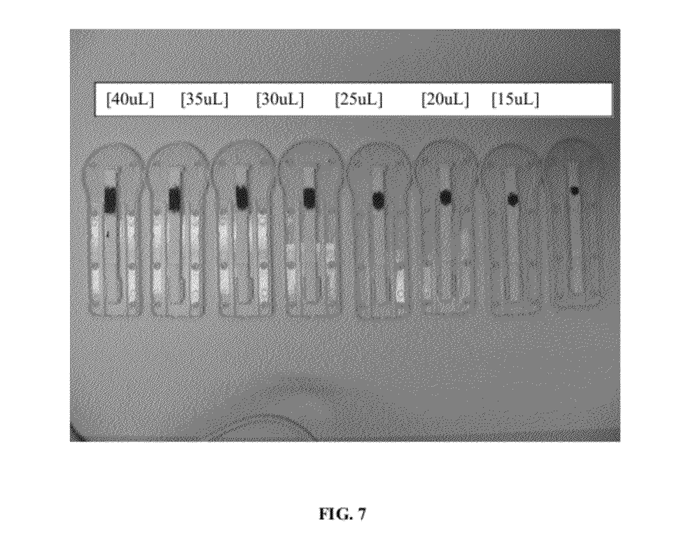

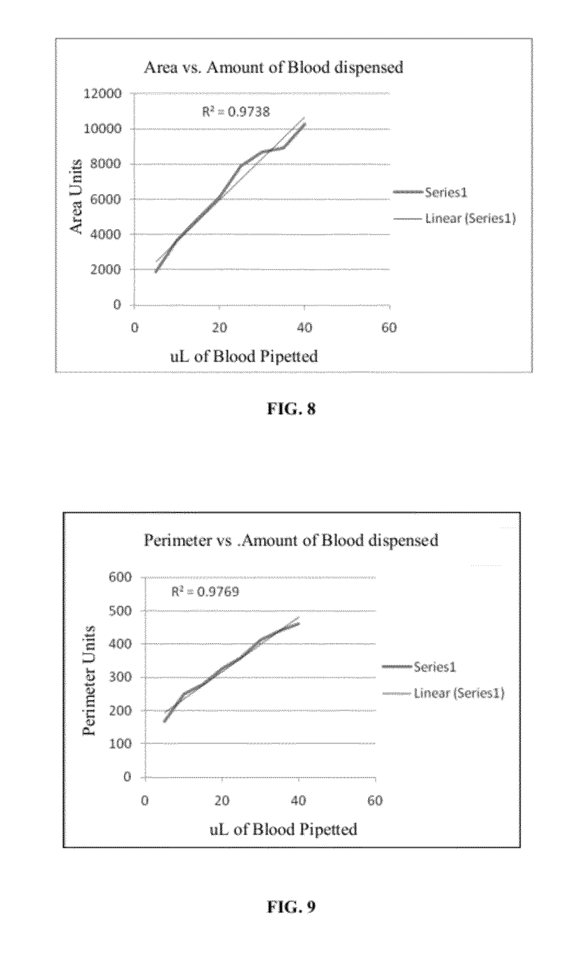

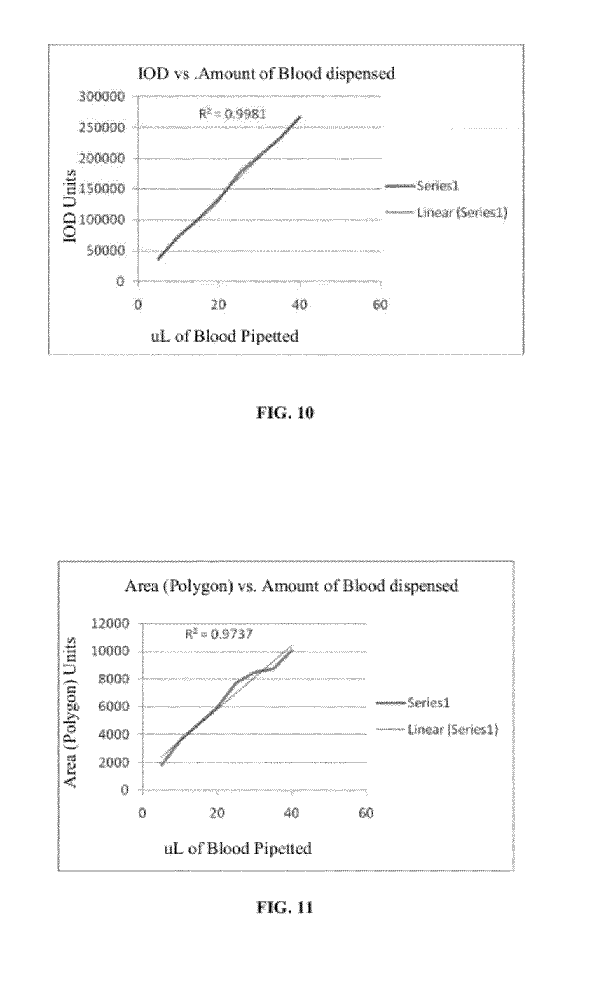

In one embodiment, the amount of sample applied to a lateral flow test strip can be determined. FIG. 7 shows significant change in spot size with sample contained to a 60 mm long ×5mm wide strip (a rectangular sample applicator). 5-40 uL of rabbit blood was pipetted onto the sample pad. In the examples shown in FIGS. 7-14 shown below, an image processing program called Image Pro Plus was used. This program has edge finding capabilities, and mimics a custom built algorithm. Table 1 below shows data produced by analyzing the test strips shown in FIG. 7.

TABLE 1

| | | | | | Area |
|---|---|---|---|---|---|
| | Obj# | Area | Perimeter | IOD | (polygon) |
| 5 uL | 2 | 1894 | 166.662 | 36314 | 1819.097 |
| 10 uL | 8 | 3687 | 249.6953 | 73450 | 3574.042 |
| 15 uL | 6 | 4930 | 278.3538 | 101044 | 4784.652 |
| 20 uL | 7 | 6129 | 325.8036 | 133434 | 5970.875 |
| 25 uL | 5 | 7911 | 359.6988 | 175867 | 7733.43 |
| 30 uL | 1 | 8701 | 412.0539 | 204233 | 8496.709 |
| 35 uL | 3 | 8930 | 439.9796 | 231622 | 8719.375 |
| 40 uL | 4 | 10292 | 459.809 | 266881 | 10067.94 |

In the table above, the software defines “Area” as “Area of object, does not include holes, if <fill holes> option is turned off” The software defines “Perimeter” as “Length of the objects outline.” IOD is defined by the software as “Integrated Optical Density” also area*average density (or intensity).” Area (polygon) is defined by the software as “area included in the polygon defining the objects outline.” The liquid spots can be analyzed in such a way to create a very accurate R² value so that when an “unknown” is encountered the volume of liquid can be calculated.

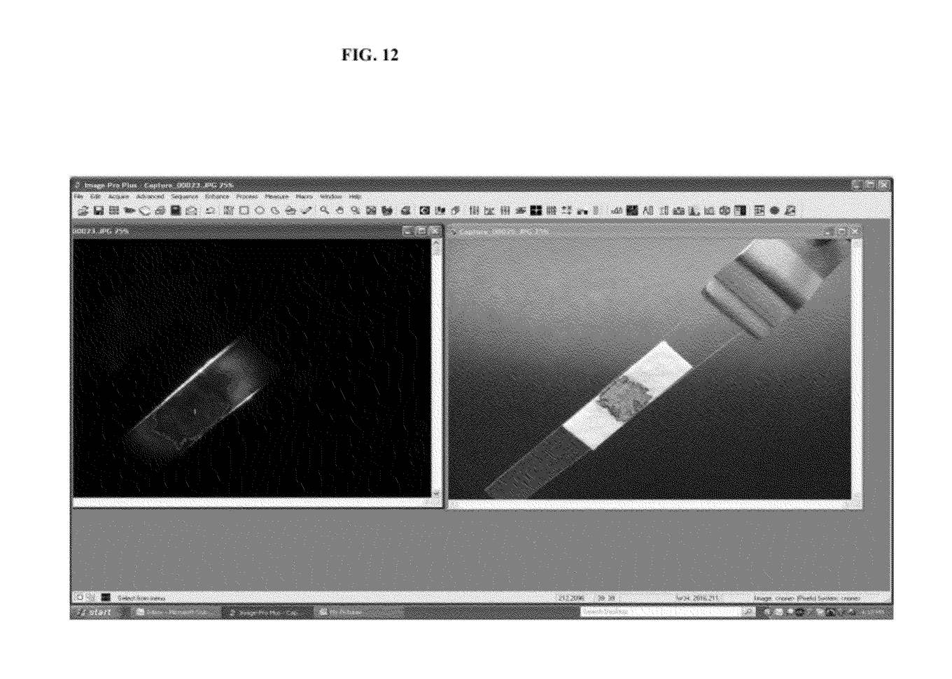

In some embodiments, transmission-based detection in which light shines through the membrane and into the camera on the other side of the blood separation pad is used. Transmission-based detection may offer more precision by measuring the amount of liquid that has absorbed past the surface of the membrane. When using reflection-based light detection, care must be taken to apply liquid to the surface of the sample application area gently to avoid having the liquid absorb down due to force, and instead absorb out on the top. If a user dispenses the liquid forcefully, then the resulting spot might be smaller than normal because most of the liquid absorbed down into the pad. Using transmission-based light detection avoids this problem. For example, a clear plastic pad backing can be used. When a light is shone from below the pad, it lights up the entire liquid spot. Dark regions indicate where liquid is present, while lighter regions indicate where no liquid is present. FIG. 12 shows the advantage of using transmission-based light detection versus reflection-based light detection. FIG. 12 shows a test strip containing 40 uL of Red dye. The image on the left shows the test strip being analyzed by transmission-based light detection. Green light is shined through the sample pad, and the image of the test strip is captured from above. The image on the right shows the test strip being analyzed by reflection-based light detection. Ambient light comes from above or around the sample, and the image of the test strip is captured from above. The image processing software performs a trace to select the object to measure, i.e. the liquid spot.

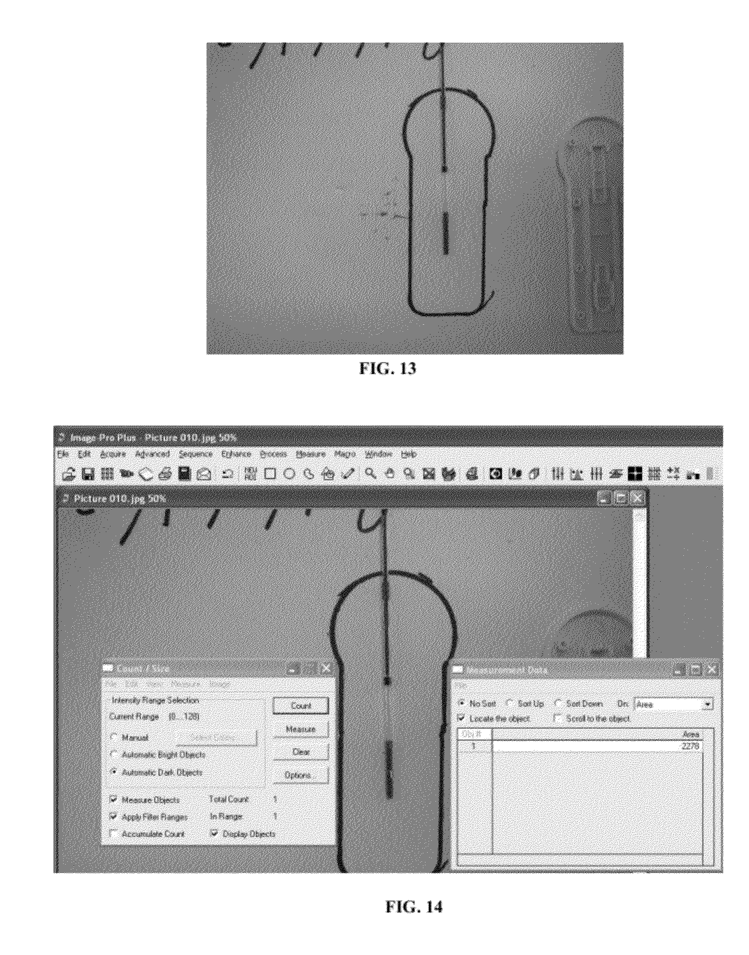

In another embodiment, the amount of sample in a capillary tube may be determined. Many diagnostic tests use a capillary tube to draw up blood from the body. In many tests, the user is instructed to draw up blood until it gets to a line indicated on the capillary tube. If the user is unable to fill the capillary tube with sample to the required level to perform the test, the test would suffer from sampling variation (i.e. either too little or too much sample). Using the image processing methods described above, the volume of sample collected and applied to the sample area can be determined, and the sample error can be corrected for. FIG. 13 shows a capillary tube containing an unknown amount of blood. FIG. 14 shows the image of FIG. 13 being processed by a software program (Image Pro Plus). The software determines the area of the object, thereby allowing the volume of sample collected to be determined.

In another embodiment, the software is programmed with a perfect dot, and, based on pixels, the software will determine the volume of the dot. The software algorithm corrects for irregular circles, which therefore corrects for volume differences. For example, if the instrument is calibrated for a value of 10, and the software returns a value of 7, the software will adjust for this difference and would reduce the sample size in the instrument accordingly. In this example, the software would add 30% to the signal that is produced. After the image has been processed and a liquid volume has been determined, this information will be used to adjust the result of the test to account for the difference in volume, and provides an “offset” based on sample volume to come up with the correct antigen amount. Immunoassays are based on antigen-antibody interactions. The more antigen present in the sample, the higher the rate of antigen-antibody interactions, and therefore a higher end test signal. For example, a 10 μL sample of blood with 100 pg/mL BNP will have a total of 1 pg BNP (0.01 mL*100 pg/mL). A 15 μL sample of the same blood will have a total of 1.5 pg BNP (0.015 mL*100 pg/mL). If the 15 μL sample was used in the test, it would generate a higher test signal then the 10 μL sample, as there are more antigen—antibody interactions. If the test is calibrated for 10 μL of sample, the 15 μL sample would cause an overestimate of the true blood sample (100 pg/mL). If the sample amount is lower than the calibrated amount, the instrument would provide an addition (+) offset. If the sample amount is more than the calibrated amount, the instrument would provide a subtraction (−) offset.

During the manufacturing of the test reagents (for example, on a lot-to-lot frequency) a curve of volume vs. antigen concentration can be generated by measuring the test signal with various amounts of antigen concentrations at various sample volumes. A table can then be generated within the test instrument using this information. The table contains the volume of sample in one column and the test signal in a second column, for each antigen concentration. Therefore each sample volume will have its own calibration curve. The test instrument can generate the slope between these points, or find the difference between the points and use this information to adjust the signal based on the volume of the sample. Once the correct calibration curve has been found, a test signal can be converted to dose (for example, a test signal of 4500 units=3.05 pg/mL BNP).

### Example 1

In this example, the instrument is calibrated for 20 μL of sample. The sample image is processed and is found to be 15.5 μL. The test instrument would use the table to determine how the test performed during manufacturing of the test at this volume. If the volume was too low to get the claimed limit of detection, the test would be rejected. If the sample was too high (for example, 40 μL instead 20 μL) then the test would also be rejected. If the sample is in the designed range, then the instrument will use the table and find the response curve needed (for example, 5 μL, 10 μL, 15 μL, 25 μL, 30 μL, etc., and calculating the slope of the curves above to find the volumes in between these values, i.e. 7 uL, 13 uL, 15.5 uL, etc.). The instrument then measures the test signal and determines the signal (i.e. 4500 units=3.05 pg/mL BNP.

1. A method of performing an assay, the method comprising:
collecting a sample;
capturing an image of the collected sample;
analyzing the image of the collected sample to determine the amount of sample collected;
determining a result of the assay; and
adjusting the result of the assay based on the amount of sample collected.

2. The method of claim 1, wherein collecting the sample comprises adding the sample to a membrane.

3. The method of claim 1, wherein collecting the sample comprises adding the sample to a test strip.

4. The method of claim 1, wherein collecting the sample comprises collecting the sample in a capillary tube.

5. The method of claim 1, wherein the image is captured using a camera.

6. The method of claim 1, wherein analyzing the image comprises using a software program to determine the volume of sample.

7. The method of claim 1, wherein analyzing the image comprises using a software program to determine the area of sample on a membrane.

8. The method of claim 1, wherein the image is analyzed using transmission-based light detection.

9. The method of claim 1, wherein the image is analyzed using reflection-based light detection.
